(19) United States
(12) Patent Application Publication
(10) Pub. No.: US 2022/0313849 A1
(43) Pub. Date: Oct. 6, 2022

(54) Sanitizing Mask Rack Apparatus

(71) Applicant: Tammy Robinson, Tampa, FL (US)

(72) Inventor: Tammy Robinson, Tampa, FL (US)

(21) Appl. No.: 17/219,950

(22) Filed: Apr. 1, 2021

(51) Int. Cl. A61L 2/10; A47B 49/00

(52) U.S. Cl. CPC A61L 2/10; A47B 49/004; A61L 2202/11

(57) ABSTRACT

A sanitizing mask rack apparatus for storing and sanitizing face masks includes a base having a base top side, a base bottom side, and a base perimeter. A stand is coupled to the base. The stand is coupled to the base top side and extends perpendicularly upwards. A carrousel is coupled to the stand. The carrousel has a carrousel inner perimeter rotatably coupled to the stand and a carrousel outer perimeter. A plurality of hooks is coupled to the carrousel. The plurality of hooks extends from the carrousel outer perimeter. Each of the plurality of hooks secures a face mask. A plurality of ultraviolet (UV) lights is coupled to the stand. The plurality of UV lights sanitizes the face masks. A power source is coupled to the base and is in operational communication with the plurality of UV lights.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to mask rack devices and more particularly pertains to a new mask rack device for storing and sanitizing face masks. The present device includes a plurality of ultraviolet (UV) sanitizing lights coupled to the stand of a rotatable rack. There may also be an additional UV light within the base of the rotatable rack.

### (2) Description of Related Art Including Information Disclosed Under 37 CFR 1.97 and 1.98

The prior art relates to mask rack devices. Known devices do not include UV sanitizing lights and are typically intended for ties or jewelry. These devices may include a cover that is entirely removable but lack covers with sliding doors.

### BRIEF SUMMARY OF THE INVENTION

An embodiment of the disclosure meets the needs presented above by generally comprising a base having a base top side, a base bottom side, and a base perimeter. A stand is coupled to the base. The stand is coupled to the base top side and extends perpendicularly upwards. A carrousel is coupled to the stand. The carrousel has a carrousel inner perimeter rotatably coupled to the stand and a carrousel outer perimeter. A plurality of hooks is coupled to the carrousel. The plurality of hooks extends from the carrousel outer perimeter. Each of the plurality of hooks is configured to secure a face mask. A plurality of ultraviolet (UV) lights is coupled to the stand. The plurality of UV lights sanitizes the face masks. A power source is coupled to the base and is in operational communication with the plurality of UV lights.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a sanitizing mask rack apparatus according to an embodiment of the disclosure.

FIG. 2 is an in-use view of an embodiment of the disclosure.

FIG. 3 is a front elevation view of an embodiment of the disclosure.

FIG. 4 is a rear elevation view of an embodiment of the disclosure.

FIG. 5 is a side elevation view of an embodiment of the disclosure.

### DETAILED DESCRIPTION OF THE INVENTION

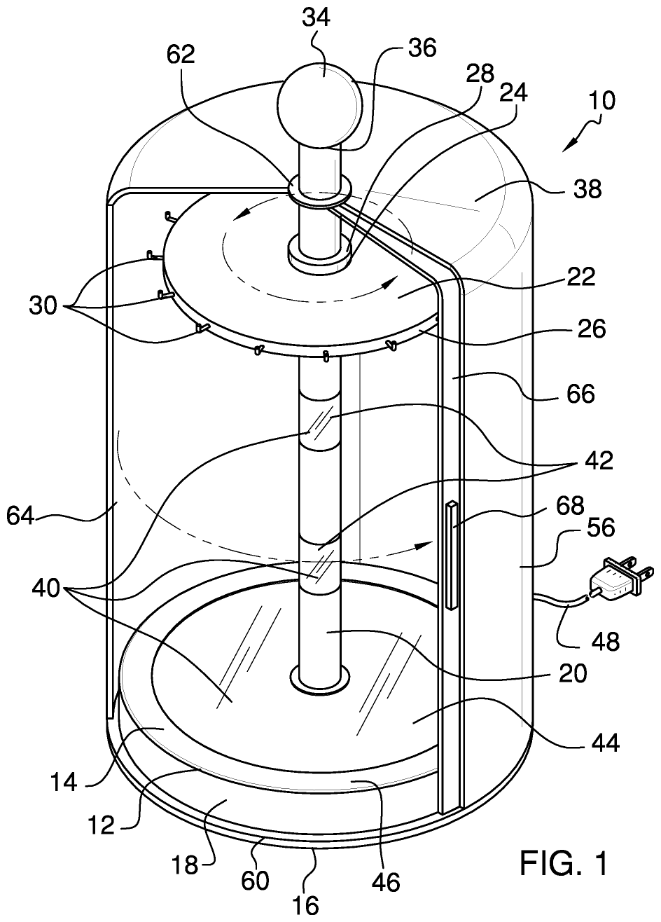

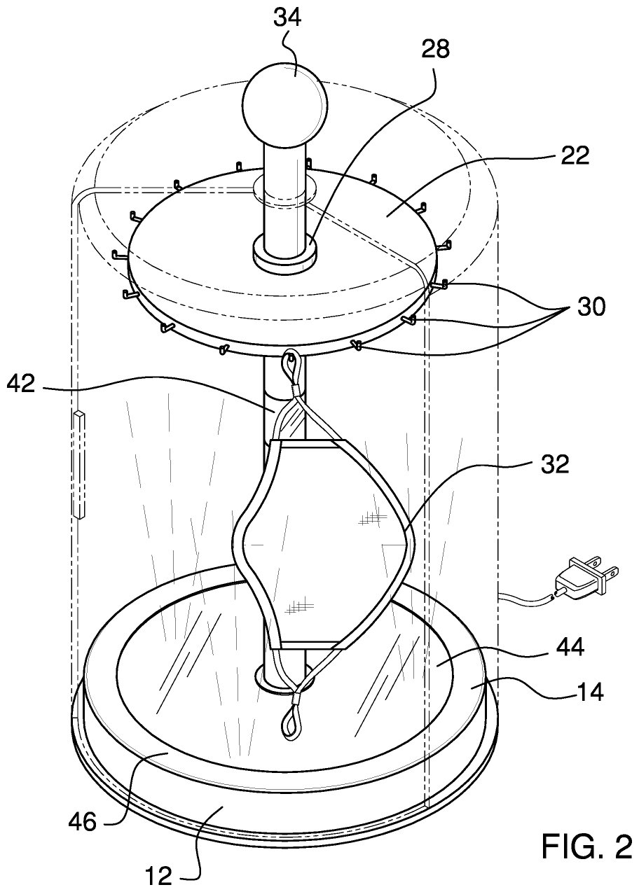

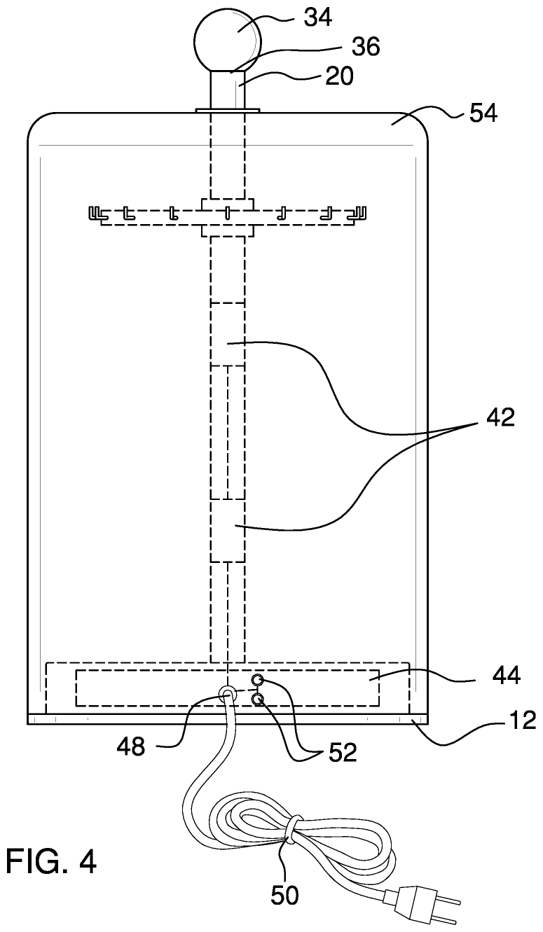

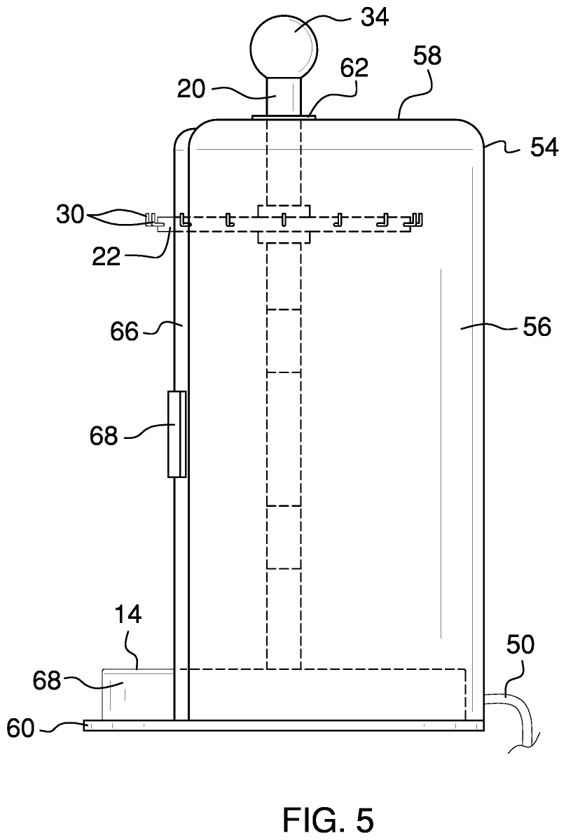

With reference now to the drawings, and in particular to FIGS. 1 through 5 thereof, a new mask rack device embodying the principles and concepts of an embodiment of the disclosure and generally designated by the reference numeral 10 will be described.

As best illustrated in FIGS. 1 through 5, the sanitizing mask rack apparatus 10 generally comprises a base 12 having a base top side 14, a base bottom side 16, and a base perimeter 18. The base 12 may be circular. A stand 20 is coupled to the base 12. The stand 20 is coupled to the base top side 14 and extends perpendicularly upwards.

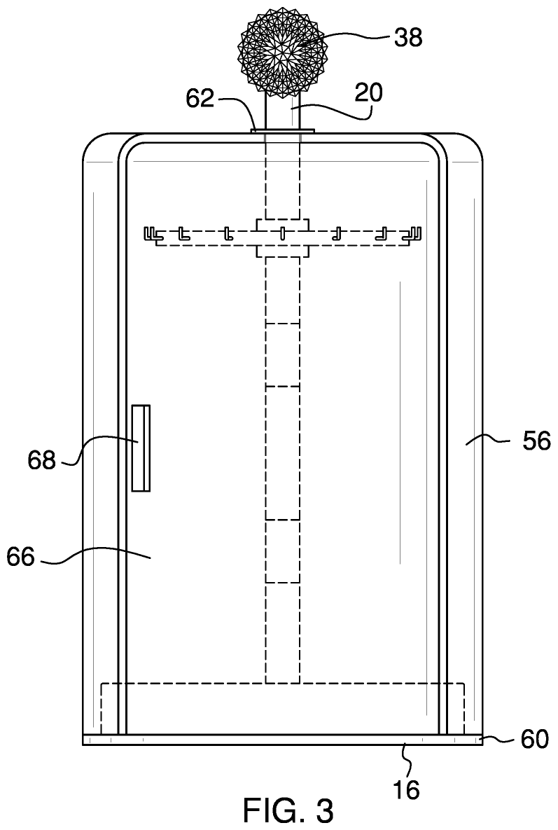

A carrousel 22 is coupled to the stand 20. The carrousel 22 has a carrousel inner perimeter 24 rotatably coupled to the stand 20 and a carrousel outer perimeter 26. The carrousel inner perimeter 24 may have a bearing 28 to provide easier rotation. A plurality of hooks 30 is coupled to the carrousel 22. The plurality of hooks 30 extends from the carrousel outer perimeter 26. Each of the plurality of hooks 30 may be L-shaped and is configured to secure a face mask 32. A finial 34 is coupled to the stand 20. The finial 34 is coupled to a stand top end 36 of the stand and may be spherical. The finial 34 may also be a crusted stone 38 as shown in FIG. 3.

A plurality of ultraviolet (UV) lights 40 is coupled to the stand 20 and the base 12. The plurality of UV lights 40 may include a pair of stand lights 42 coupled to the stand 20 and a base light 44 coupled to the base 12. The base light 44 may be circular and may occupy all of the base top side 14 excepting an outer rim portion 46 adjacent the base perimeter 18. Each of the pair of stand lights 42 may be cylindrical and fully extend around the stand 20. The plurality of UV lights 40 sanitizes the face masks 32 between uses.

A power source 48 is coupled to the base 12. The power source 48 is in operational communication with the plurality of UV lights 40. The power source 48 may be a power cord 50 or may alternatively be a battery. A pair of indicator lights 52 may be coupled to the base perimeter 18. The pair of indicator lights 52 is in operational communication with the plurality of UV lights 40 to signal to a user whether or not the face masks 32 have been sufficiently sanitized.

A cover 54 is coupled to the base 12 and the stand 20. The cover 54 has a cover wall 56 coupled to the base perimeter 18 and a cover top side 58 coupled to the stand 20. The cover 54 may rest on an outer lip portion 60 of the base perimeter 18 adjacent the base bottom side 16. The cover top side 58 may have a top ring 62 surrounding the stand 20 between the pair of stand lights 42 and the finial 34. The finial 34 may be selectively removable to allow the top ring 62 to be slid over the stand top end 36 to remove the cover 54 from the base 12. The cover 54 has a door aperture 64 and a cover door 66 rotatably covering and exposing the door aperture 64. The door aperture 64 may occupy between 120° and 180° of the cover wall 56 and the cover top side 58 and may extend from the top ring 62 to adjacent the outer lip portion 60 of the base perimeter. The cover door 66 may have a handle protrusion 68 to avoid over rotation behind the cover wall 56.

In use, the face mask 32 is hung on the plurality of hooks 30 and the cover door 64 is rotated to cover the door aperture 62. The power source 48 is activated to turn on the plurality of UV lights 40 and sanitize the face mask 32. The pair of indicator lights 52 may be observed to see when the face mask 32 has been fully sanitized before the next use.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sanitizing mask rack apparatus comprising:
a base having a base top side, a base bottom side, and a base perimeter;
a stand coupled to the base, the stand being coupled to the base top side and extending perpendicularly upwards;
a carrousel coupled to the stand, the carrousel having a carrousel inner perimeter rotatably coupled to the stand and a carrousel outer perimeter;
a plurality of hooks coupled to the carrousel, the plurality of hooks extending from the carrousel outer perimeter, each of the plurality of hooks being configured to secure a face mask;
a plurality of ultraviolet (UV) lights coupled to the stand, the plurality of UV lights sanitizing the face masks; and
a power source coupled to the base, the power source being in operational communication with the plurality of UV lights.

2. The sanitizing mask rack apparatus of claim 1 further comprising the plurality of UV lights including a pair of stand lights coupled to the stand and a base light coupled to the base.

3. The sanitizing mask rack apparatus of claim 2 further comprising the base light being circular and occupying all of the base top side excepting an outer rim portion adjacent the base perimeter.

4. The sanitizing mask rack apparatus of claim 2 further comprising the pair of stand lights being cylindrical and fully extending around the stand.

5. The sanitizing mask rack apparatus of claim 1 further comprising a finial coupled to the stand, the finial being coupled to a stand top end of the stand.

6. The sanitizing mask rack apparatus of claim 5 further comprising the finial being spherical.

7. The sanitizing mask rack apparatus of claim 5 further comprising the finial being a crusted stone.

8. The sanitizing mask rack apparatus of claim 1 further comprising a cover coupled to the base and the stand.

9. The sanitizing mask rack apparatus of claim 1 further comprising the cover having a cover wall coupled to the base perimeter and a cover top side coupled to the stand.

10. The sanitizing mask rack apparatus of claim 9 further comprising the cover having a door aperture and a cover door rotatably covering and exposing the door aperture.

11. The sanitizing mask rack apparatus of claim 9 further comprising the door aperture occupying between 120° and 180° of the cover top side.

12. The sanitizing mask rack apparatus of claim 1 further comprising each of the plurality of hooks being L-shaped.

13. The sanitizing mask rack apparatus of claim 1 further comprising the carrousel inner perimeter having a bearing.

14. A sanitizing mask rack apparatus comprising:
a base having a base top side, a base bottom side, and a base perimeter;
a stand coupled to the base, the stand being coupled to the base top side and extending perpendicularly upwards;
a carrousel coupled to the stand, the carrousel having a carrousel inner perimeter rotatably coupled to the stand and a carrousel outer perimeter, the carrousel inner perimeter having a bearing;
a plurality of hooks coupled to the carrousel, the plurality of hooks extending from the carrousel outer perimeter, each of the plurality of hooks being L-shaped and configured to secure a face mask;
a finial coupled to the stand, the finial being coupled to a stand top end of the stand, the finial being spherical;
a plurality of ultraviolet (UV) lights coupled to the stand and the base, the plurality of UV lights including a pair of stand lights coupled to the stand and a base light coupled to the base, the base light being circular and occupying all of the base top side excepting an outer rim portion adjacent the base perimeter, the pair of stand lights being cylindrical and fully extending around the stand, the plurality of UV lights sanitizing the face masks;
a power source coupled to the base, the power source being in operational communication with the plurality of UV lights; and
a cover coupled to the base and the stand, the cover having a cover wall coupled to the base perimeter and a cover top side coupled to the stand, the cover having a door aperture and a cover door rotatably covering and exposing the door aperture, the door aperture occupying between 120° and 180° of the cover top side.
